(19) United States
(12) Patent Application Publication
(10) Pub. No.: US 2008/0249456 A1
(43) Pub. Date: Oct. 9, 2008

(54) Pulsation-type auxiliary circulation system, pulsatile flow generation control device, and pulsatile flow generation control method

(76) Inventors: Syuuji Inamori, Minoo-shi (JP); Chikao Uyama, Mishima-gun (JP)

Correspondence Address: Quinn Emanuel Urquhart Oliver & Hedges, LLP;Koda/Androlia, 10th Floor, 865 S. Figueroa Street, Los Angeles, CA, 90017, US

(21) Appl. No.: 12/079,282

(22) Filed: Mar. 26, 2008

(30) Foreign Application Priority Data
Mar. 27, 2007 (JP) 2007-82888
Mar. 10, 2008 (JP) 2008-59331

(51) Int. Cl. A61M 1/36

(52) U.S. Cl. 604/6.1; 604/6.14

(57) ABSTRACT

Pulsatile flow in synchronization with the systole and diastole of a patient's own pulse is generated during auxiliary circulation therapy administered to a patient under cardiac dysfunction. As a result, the diastole augmentation function is maintained, blood flow to the coronary artery is increased, and the therapeutic effect of intending to restore cardiac function without applying after-loading to the heart is improved.

Pulsatile flow generation control device Y incorporated in a pulsation-type auxiliary circulation system (X) as an apparatus system having an electromagnetic valve 7 in blood supply lines (4, 6, 8) on the delivery side of artificial lung 5 and a control means 11 for performing control-output relating to the opening and closing operation of the electromagnetic valve 7. The control means 11 has at least a delay circuit and is such that the electromagnetic valve 7 is actuated to close and is maintained closed for a predetermined time interval based on delay detection and is actuated to open based on delay cancellation so that blood flow is periodically interrupted.

### BACKGROUND OF THE INVENTION

1. Field of the Invention

The present invention relates to a blood circulation system having a blood circulation path, which is externally connected to a human body in cardiac dysfunction (a patient) and that comprises an blood removal line and a blood supply line, and a centrifugal pump, which is for using the blood circulation path as an auxiliary circulation path or for circulating blood between a human body and an artificial lung; and more specifically the present invention relates to a pulsation-type auxiliary circulation system, a pulsatile flow generation control device, and a pulsatile flow generation control method that conducts or promotes heart disease therapy (auxiliary circulation therapy) by generating pulsatile flow in synchronization with the systole and diastole of the patient's own pulse based on measurement of electrocardiogram waveform and/or pressure waveform.

The phrase “pulsation-type auxiliary circulation system” used in the present application means an apparatus system that includes a pulsatile flow generation control device.

2. Description of the Related Art

Today there are a variety of methods for treating cardiac dysfunction depending on the severity of the illness. In mild cases, the patient is instructed to make lifestyle changes or is put on pharmacotherapy, while in severe cases intra-arterial balloon pumping (IABP), percutaneous pulmonary support (PCPS hereafter), or a high-performance medical device such as an artificial heart is used. A variety of existing technologies can be referred to when a summary of blood circulation system is expanded to include the artificial heart-lung for surgery (for instance, refer to Patent References 1, 2, 3, 4, 5, 6, 7, 8, and 9 below).

[Patent Reference 1] Japanese Patent Application Laid-Open (Kokai) No. H6-63124

[Patent Reference 2] Japanese Patent Application Laid-Open (Kokai) No. H110-76002

[Patent Reference 3] Japanese Patent Application National Publication (Kohyo) No. 2000-508950

[Patent Reference 4] Japanese Patent Application National Publication (Kohyo) No. 2001-508669

[Patent Reference 5] Japanese Patent Application Laid-Open (Kokai) No. 2002

[Patent Reference 6] Japanese Patent Application Laid-Open (Kokai) No. 2004-97611

[Patent Reference 7] Japanese Patent Application Laid-Open (Kokai) No. 2007-43436

[Patent Reference 8] Japanese Patent Application Laid-Open (Kokai) No. 2006

[Patent Reference 9] Japanese Patent Application Laid-Open (Kokai) No. 2007-44302

Of such devices and methods, auxiliary circulation (PCPS) is frequently the first choice for use against cardiac dysfunction attributed to cardiogenic shock accompanying acute myocardial infarction or severe acute myocarditis, and the like.

For instance, when auxiliary circulation is provided for the purpose of saving a life and restoring the cardiac function of a patient in cardiac dysfunction due to various causes, the therapeutic time is determined by the patient's status and the therapeutic results; however, therapy generally takes a long time (from several days to several weeks) when compared to the extracorporeal circulation by the artificial heart-lung (several hours) that is generally used for heart surgery. Therefore, supplying blood using a roller-pump artificial heart-lung is inappropriate because it induces hemoclasis. For these reasons, the current auxiliary circulation therapy generally uses a centrifugal pump to supply blood. However, when the cardiac function of a patient is greatly diminished, the problem is that the centrifugal pump which generates a steady flow is unable to supply pulsatile flow systemically (discussed later). In this state, though the systemic blood flow is maintained, the protective effect on individual organs is insufficient. In other words, the effect of the diastolic augmentation function on the heart cannot be expected.

As previously mentioned, the supply of blood by a centrifugal pump is generally a non-pulsatile flow. In contrast to this, physiologically the heart itself supplies pulsatile flow systemically. This supply-effect is an extremely important function in maintaining systemic circulation and protecting each organ. The maintenance of pulsatile flow to the heart is particularly necessary because the heart has the diastolic augmentation function of maintaining the amount of blood flowing to the coronary artery by the pulsatile flow of the heart itself.

Nevertheless, there are often no alternatives but to use the steady flow because of the fact that the current PCPS systems are indicated for a state of cardiac dysfunction and because a centrifugal pump cannot generate a pulsatile flow.

Furthermore, continuous perfusion by a steady flow runs the risk of an increase in the after-load on the heart during the recovery process and has the opposite effect during treatment intended to restore cardiac function. In the past, pulsatile flow has been obtained using a generator that operates by the turning of a roller pump in the artificial heart-lungs that are used as auxiliary means during heart surgery, or by blowing out and in the gas of a balloon left in the blood vessels of a patient as seen with an intra-arterial balloon pumping (IABP) apparatus; however, neither of these are satisfactory because they pose problems in terms of indications, invasiveness, and cost.

### BRIEF SUMMARY OF THE INVENTION

The problem to be solved by the invention is the fact that the auxiliary circulation therapy administered to a patient under the stress of a state of cardiac dysfunction is not the therapy that is indicated for a patient in a state of cardiac arrest as is the artificial heart-lung; instead, since it is used while the patient's heart is functioning, pulsatile flow synchronized with the patient's heart rhythm must be supplied.

The synchronized timing here requires that it be possible to detect the electrocardiogram R wave and adjust the timing for actuation at the beginning of the electrocardiogram T wave at a predetermined time interval, rather than for actuation in synchronization with the electrocardiogram R wave of a patient itself as with a conventional defibrillator.

The present invention is made in light of these circumstances, and an object of the present invention is to solve the above-described problems and provide a pulsation-type auxiliary circulation system, a pulsatile flow generation control device, and a pulsatile flow generation control method, in which, by means of auxiliary circulation therapy that is continuously provided to a patient under the stress of a state of cardiac dysfunction, it is possible to supply pulsatile flow to the body and thereby maintain the diastolic augmentation function and improve the therapeutic effect intended to restore cardiac function without increasing the coronary artery blood flow or placing the heart under after-loading.

In order to solve the problems, the present invention is a pulsation-type auxiliary circulation system used in a blood circulation system and characterized in that the system comprises
a blood circulation path that is externally connected to a human body in cardiac dysfunction (a patient) and comprised of a blood removal line and a blood supply line; and
a centrifugal pump for using the blood circulation path as an auxiliary circulation path or for circulating blood between a human body and an artificial lung; and wherein
the pulsation-type auxiliary circulation system conducts or promotes heart disease therapy by generating pulsatile flow in synchronization with the systole and diastole of the patient's own pulse based on measurement of the electrocardiogram waveform and/or pressure waveform, and comprises
   an electromagnetic valve disposed in the blood supply line so as to be on a delivery side of the artificial lung,
   a control unit for performing control-output relating to opening and closing operation of the electromagnetic valve, and
   a biological signal monitoring device that is connected to the control unit and that is capable of measuring and displaying biological signals including at least the electrocardiogram waveform and pressure waveform; and
the control unit has a pulsatile flow generation control means for periodically interrupting a supply of blood by maintaining the electromagnetic valve closed for a predetermined time interval via at least a delay circuit.

Moreover, in the above-described blood circulation system or pulsation-type auxiliary circulation system, the present invention is characterized in that
the pulsatile flow generation control device is a pulsatile flow generation control device wherein auxiliary circulation therapy is conducted or promoted by generating pulsatile flow in synchronization with systole and diastole of patient's own pulse based on measurement of electrocardiogram waveform and/or pressure waveform; and
it comprises
   an electromagnetic valve disposed in the blood supply line so as to be on a delivery side of an artificial lung, and
   a control means for performing control-output relating to opening and closing operation of the electromagnetic valve; and
   the control means has at least a delay circuit and actuates the electromagnetic valve to close based on delay detection, maintains the electromagnetic valve closed for a predetermined time interval, and actuates the magnetic valve to open based on delay cancellation such that a supply of blood is periodically interrupted.

Moreover, the present invention is a pulsatile flow generation control method for a pulsation-type auxiliary circulation system, and it is characterized in that the pulsatile flow generation control method performs control-output in which the electrocardiogram waveform obtained from patient is amplified, electrocardiogram R waveform is detected as a trigger by waveform shaping therapy, a starting point is inputted to a delay circuit, an electromagnetic valve is actuated to close and is maintained closed for a predetermined time interval based on delay detection, and the electromagnetic valve is actuated to open based on delay cancellation.

According to the present invention, it becomes possible to supply a physiological pulsatile flow to a patient who has entered severe cardiac dysfunction, enhancing the auxiliary circulation effect.

More specifically, by supplying pulsatile flow to the body during auxiliary circulation therapy administered to a patient under the stress of a state of cardiac dysfunction, the diastolic augmentation function is maintained and the coronary artery blood flow is increased. Moreover, selective auxiliary circulation in the diastole becomes possible; and as a result, it is possible to maintain the P+ΔP pressure-elevating effect on the diastolic pressure P of the heart itself. An increase in coronary artery blood flow and restoration of cardiac function can be expected as a result of this secondary effect. Therefore, it is possible to improve the therapeutic effect of intending to restore cardiac function without applying after-loading to the heart during continuous auxiliary circulation therapy.

### BRIEF DESCRIPTION OF THE SEVERAL VIEWS OF THE DRAWINGS

FIG. 1 is an explanatory diagram of the pulsation-type auxiliary circulation system of the present invention;

FIG. 2 is an illustration showing the control box (control unit) of the operating system of the present invention;

FIG. 3 is an explanatory diagram of the apparatus of the embodiment (pulsatile flow generation control device);

FIG. 4 is a flow sheet (flow chart) showing the control means (procedure) of the apparatus of the embodiment (pulsatile flow generation control device);

FIG. 5 is a time chart showing the time relationship of the electrocardiogram waveform and electromagnetic valve opening and closing operation in the apparatus of the embodiment (pulsatile flow generation control device);

FIG. 6 is a circuit diagram of the system of Embodiment 2 of the present invention (including pulsatile flow generation control device);

FIG. 7 is a graph showing the changes in the CPK-MB value from the experiment of the present invention; and

FIG. 8 is an illustration showing the pulsatile flow myocardial protection experimental circuit in Embodiment 3.

### DETAILED DESCRIPTION OF THE INVENTION

In the preferred embodiment of the present invention, the control means of the pulsatile flow generation control device having the above-described structure includes an electrocardiogram waveform amplification circuit, a waveform shaping circuit, a delay circuit, an electromagnetic valve current on/off control circuit 17, and a device for opening and closing the electromagnetic valve; and this control means performs control-output for inputting the electrocardiogram R waveform detected from the amplified electrocardiogram waveform as the starting point and performing delay detection; for actuating the electromagnetic valve to close based on the delay detection; for maintaining the electromagnetic valve closed for a predetermined time interval; and for actuating the electromagnetic valve to open based on delay cancellation.

Moreover, by means of the pulsation-type auxiliary circulation system, pulsatile flow generation control device, and pulsatile flow generation control method having the above-described structure, the predetermined time interval is the delay time setting of the delay circuit that performs the detection and cancellation for blocking of the blood supply line by electromagnetic valve operation; this delay time setting is the time from when delay is detected with the threshold electrocardiogram R waveform as the starting point until when delay is canceled with the beginning of electrocardiogram T waveform generation as the ending point; and this delay cancellation can be adjusted while monitoring the dichrotic notch of the pressure waveform, which is the distinguishing waveform when the aortic valve is obstructed.

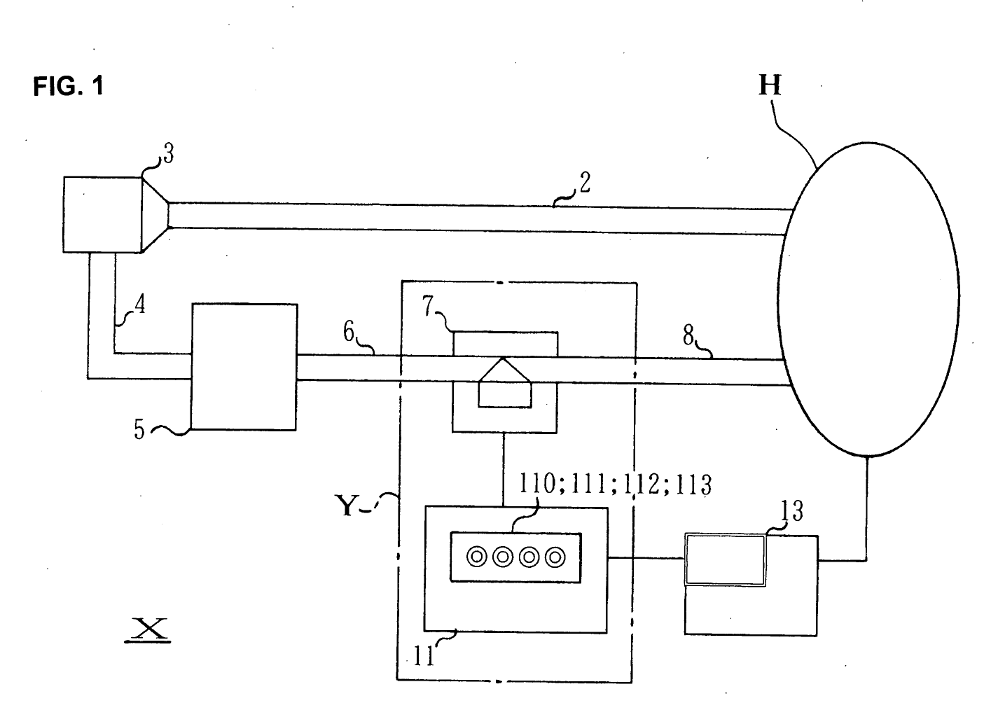

FIG. 1 is an explanatory diagram of the structure of the pulsation-type auxiliary circulation system of the present invention, and this embodiment will now be described in specific terms.

As shown in the figure, the pulsation-type auxiliary circulation system X is to act as a substitute for the severely diminished cardiac function of a patient in order to save the patient's life and restore cardiac function.

The procedure involves connecting of a blood removal tube 2 (venous blood tube) to the indwelling blood removal tube in a vein of a patient to construct a blood removal line and connecting of a blood supply tube 8 (arterial blood tube) to an indwelling blood supply tube in the artery of the patient to construct a blood supply line.

Negative pressure is produced in the blood removal tube 2 (blood removal line) by turning centrifugal pump 3, thus guiding venous blood of the patient up to the centrifugal pump 3. There the venous blood, which has been brought to positive pressure, is introduced to artificial lung 5 and oxygenated to become arterial blood, and this arterial blood is supplied to the patient through the blood supply tube 8 (blood supply line).

A small electromagnetic valve 7 is attached to the blood supply tube 8 (blood supply line) in order to generate pulsatile flow during this series of processes; and by repeatedly pressing to close and then releasing to open this blood supply tube 8 (including elastic blood supply tube 6), pulsatile flow is administered to the patient.

How the magnetic valve is actuated can be selected from the specific-number-of-times system (which is used primarily in emergency, when the electrocardiogram waveform of a patient cannot be obtained) and the synchronized system.

The optimal method for driving the magnetic valve is the synchronized system, in which since the diastolic augmentation function works in the diastole of the heart, blood supply tube 8 (blood supply line) is released to open by the magnetic valve 7 at the beginning of the T wave on the electrocardiogram waveform.

Accordingly, such a setting is established that the electrocardiogram waveform of the patient is obtained from EKG monitor 13, the R wave is detected, and the electromagnetic valve is, using a delay timer (delay circuit), actuated in alignment with (in synchronization with) the systole and diastole of the patient's own pulse.

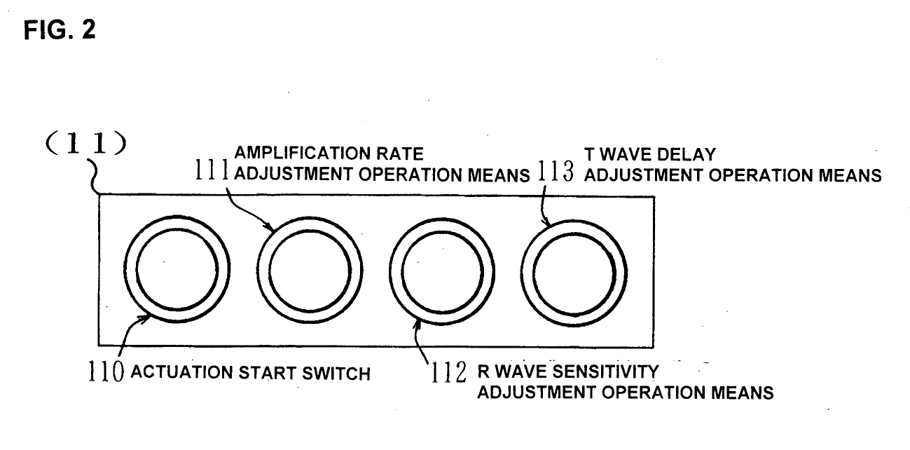

FIG. 2 shows the control unit operating system. In order to realize the effect of the diastolic augmentation function, the control box (control unit 11) is provided therein a drive initiation switch 110, an amplification rate adjustment operation means 111 for variable adjustment of the electrocardiogram waveform amplification rate, an R wave sensitivity adjustment operating switch 112 for the purpose of aligning the number of times the electromagnetic valve 7 is actuated with the pulse of the patient (number of heart beats), and a T wave delay adjustment operating means 113 for opening the electromagnetic valve 7 at the beginning of the T wave on the electrocardiogram waveform and bringing the blood supply tube 8 (blood supply line) to an open state.

The blood removal tube 2 and the blood supply tube 8 (including elastic blood supply tube 6) for extracorporeal circulation are elastic tubes; and for these tubes, a resin material having relatively high elasticity, preferably polyvinyl chloride, is used for the purpose of preventing clogging and bending. The reason for this is that even if the tubes are press-closed by the electromagnetic valve 7, when the switch is turned off, the tube (the blood supply tube 8) can quickly recover to an open state because of this elasticity.

More specifically, with the above arrangement, it is possible to provide a physiological pulsatile flow, even for the auxiliary circulation that operates under a steady flow because of successive press-closing of the tube by the electromagnetic valve 7 and opening of the tube by its own elasticity.

The setting of the delay timer (delay circuit) will now be described below.

Under a state of physiological circulatory dynamics, the blood flow of the coronary artery, which is the nourishing blood vessel of the heart, primarily takes on a state of being drawn into the coronary artery by the negative pressure effect of the diastole rather than being forced into the systole of the heart.

Consequently, there are cases in which sufficient pulsation is not generated in a state of cardiac dysfunction and the diastolic augmentation function is not effective. In other words, when blood flow can be selectively provided by auxiliary circulation to the diastole of the heart, the diastolic blood pressure will rise, and as a result, an effective means for increasing the blood flow to the coronary artery and restoring cardiac function is provided.

Therefore, the delay timer is set such that so as for the release-opening point of the electromagnetic valve 7 to correspond to the diastole of the patient, the R wave is detected from the electrocardiogram waveform of the patient, and the electromagnetic valve 7 is, using the T wave delay adjustment means 113, opened at the beginning of the T wave, which is indicative of the diastole of the patient.

In summarizing the above-described function, the centrifugal pump 3 turns by a magnetic transmission system when auxiliary circulation is conducted. This turning produces negative pressure in the blood removal tube 2 and introduces venous blood of the patient to the blood removal tube 2 (blood removal line) side. The venous blood is suctioned by the centrifugal pump 3 via the blood removal tube 2 (blood removal line) and here pressure becomes positive pressure and the blood is supplied to the artificial lung 5.

The blood that has been oxygenated by the artificial lung 5 becomes arterial blood and is supplied through the blood supply tube 8 (blood supply line) to the artery of the patient. However, the centrifugal pump 3 is a device that generates a steady flow and cannot administer the physiological pulsatile flow of the body.

Therefore, in the present invention, the simple electromagnetic valve 7 is provided on the blood supply side so that a pulsatile flow is imparted to the patient's body by mechanically pressing to close and then releasing to open the blood supply tube 8 (blood supply line) in succession.

The electromagnetic valve 7 is controlled by the control box 11 and is opened and closed in accordance with the pulse (heart beat) of the patient.

Ideally, in order to make auxiliary circulation therapy effective, as long as the electrocardiogram waveform of a patient can be measured, it is done to find the R wave from a monitor and then adjust the time when the electromagnetic valve 7 presses to close and releases to open the blood supply tube 8 such that the electromagnetic valve 7 releases to open the tube 8 at the beginning of the T wave, which shows the diastole of the heart.

### Embodiment 1

A pulsatile flow generation control device that is one example of the present invention (example apparatus) will now be described below with reference to the drawings.

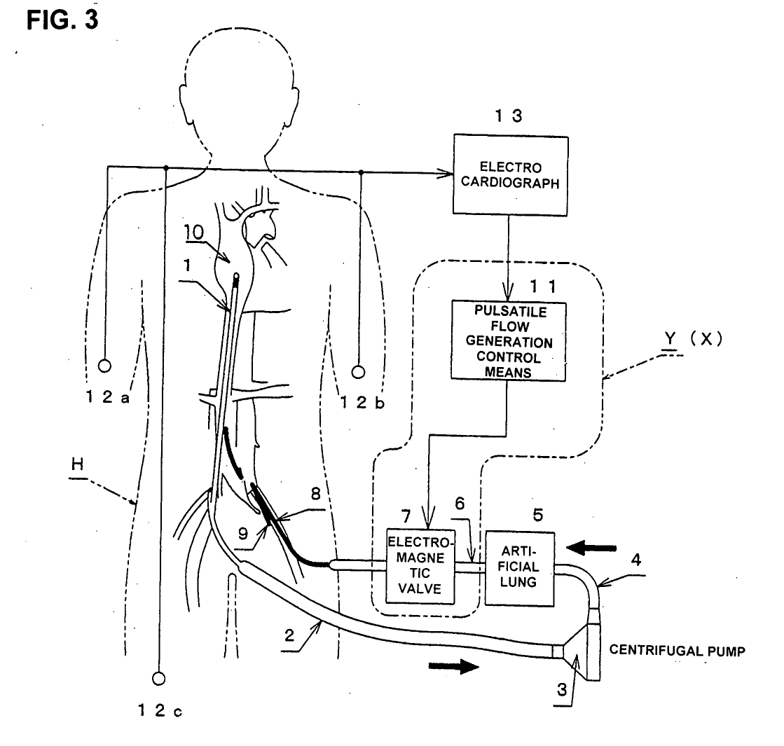

FIG. 3 is an explanatory diagram of the structure of the apparatus of the Embodiment.

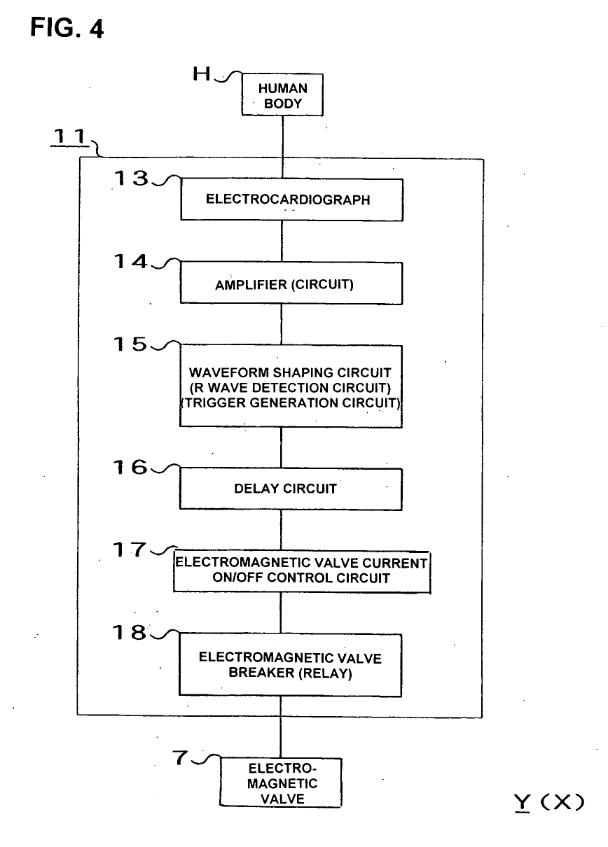

FIG. 4 is the flow sheet (flow chart) showing the control means (procedure) of the apparatus of the Embodiment.

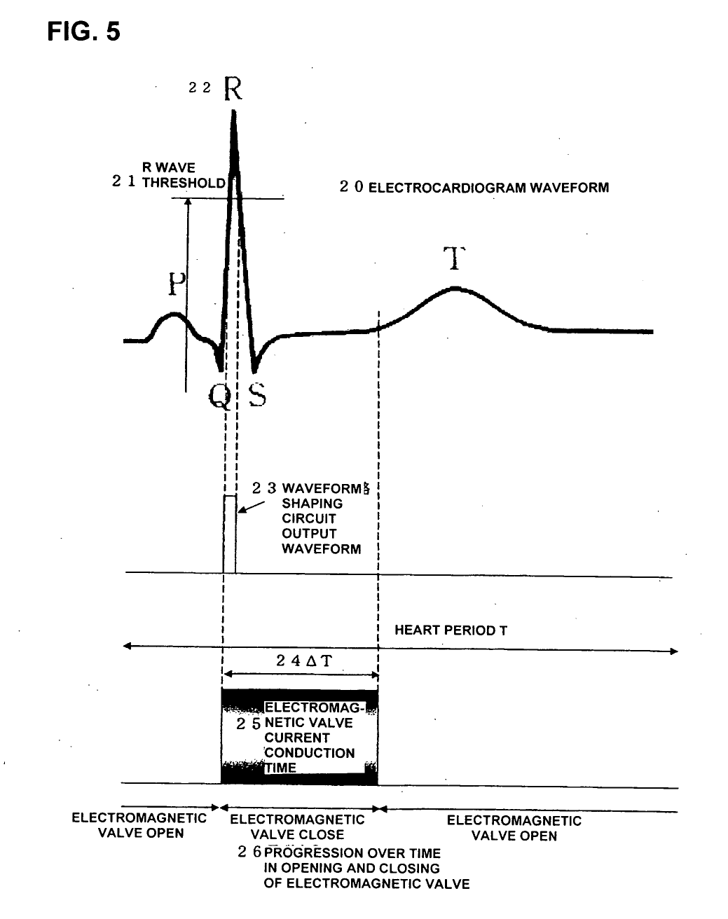

FIG. 5 is a time chart showing the time relationship between the electrocardiogram waveform and the electromagnetic valve opening and closing operation in the apparatus of the Embodiment.

As illustrated, the apparatus of the Embodiment X includes an electromagnetic valve 7 disposed in the blood supply line (8) on the delivery side of the artificial lung 5, a control means 11 for executing control-output related to the opening and closing operation of the electromagnetic valve 7, and a biological signal monitor 13 (an electrocardiograph in the drawing) that is connected to the control means 11 and is capable of measuring and displaying biological signals including at least the electrocardiogram waveform and pressure waveform; and in this apparatus, the control means 11 performs control-output for periodically interrupting the supply of blood by maintaining the electromagnetic valve 7 closed for a predetermined time interval via at least a delay circuit 16.

Here, the control means 11 is provided with an electrocardiogram waveform amplification circuit 14, a waveform shaping circuit 15 (including an R wave detection circuit and trigger generation circuit), a delay circuit 16, an electromagnetic valve current on/off control circuit 17, and a device 18 for turning the electromagnetic valve on and off. The control means 11 performs control-output so that it inputs the trigger (input the starting point) of the electrocardiogram R wave 22, which has been detected by waveform wave shaping from the amplified electrocardiogram waveform, and performs delay detection; and it further actuates the electromagnetic valve 7 to close based on this delay detection and maintain the valve closed for a predetermined time interval via the delay circuit 16 and actuates the electromagnetic valve 7 to open based on delay cancellation.

The predetermined time interval is the delay time setting of the delay circuit 16, which performs the detection and cancellation for blocking the blood supply line 8 by operation of the electromagnetic valve 7. This delay time setting is the time from when delay is detected with the threshold electrocardiogram R waveform 21 (22) as the starting point until the delay is cancelled with the beginning of the generation of the electrocardiogram T waveform as the ending point, and this delay cancellation can be adjusted by monitoring the dichrotic notch (not illustrated in the drawings) of the pressure waveform, which is the distinguishing waveform during aortic dilation.

The specific structure is described in further detail below. As shown in FIG. 3, the tip of blood removal tube 1, which has been inserted from the right femoral vein of patient H, is retained indwelling in the sinus venarum cavarum or right atrium 10 and blood is guided through the venous blood supply tube 2 to the centrifugal pump 3. The blood guided to the centrifugal pump 3 receives centrifugal force from the turning of a rotating body of the centrifugal pump 3 to which the blood contacts and is then guided through the coupling tube 4 to the artificial lung 5. The blood that has been oxygenated by the artificial lung 5 is sent to the elastic blood supply tube 6, goes through the valve port of the electromagnetic valve 7, and returns to the left femoral artery 9 of the patient through the arterial blood supply tube 8. Opening and closing of the electromagnetic valve 7 is controlled by the pulsatile flow generation control means 11. Electrocardiogram waveform (20) (see FIG. 5) obtained at electrocardiograph electrodes 12a, 12b and 12c is sent to the electrocardiograph 13 and the signals are sent to the pulsatile flow generation control means 11 as electrocardiogram waveform (20) is being drawn on the monitor screen. R wave 22 (21), which is the ventricular systole signal, is detected from the electrocardiogram waveform (20) by the pulsatile flow generation control means 11; and beginning at this detected time, the blood that has been supplied through the arterial blood supply tube 8 is stopped by closing the electromagnetic valve 7 for time ΔT, which corresponds to the ventricular systole. Once the time of ΔT has passed, the electromagnetic valve 7 is opened and the supply of blood through the arterial blood supply tube 8 is re-started. The time when blood is fed by the opening and closing of the electromagnetic valve 7 corresponds to exactly the ventricular diastole, and a diastolic augmentation effect can be expected.

As shown in FIGS. 4 and 5, in order to realize a diastolic augmentation effect, blood flow via the pulsatile flow generation control means 11 is stopped during ventricular systole from the beginning of R wave 22 to until immediately before the start of the T wave; and when systole ends, this blood is supplied through the pulsatile flow generation control means 11. Therefore, signals from the electrocardiograph 13 are amplified by being inputted to the amplification circuit 14, which amplifies the electrocardiogram waveform 20 to appropriate voltage. The R wave 22 (21) is detected from the output signal (20) of this amplification circuit 14; therefore, the amplified signal 20 is inputted to the waveform shaping circuit 15. It is possible to determine the R wave 22 starting time by appropriately setting R wave threshold 21 at electrocardiogram waveform signal 20 and generating trigger waveform 23, which corresponds to the time interval of R wave 22, whose starting point is this R wave 22 starting time. Pulse signal (25) of variable time interval ΔT24 is outputted at the delay circuit 16 with this trigger waveform 23 as the starting time. Time interval ΔT24 of pulse signal (25) from this delay circuit 16 is inputted to the electromagnetic valve current on/off control circuit 17. The electromagnetic valve current on/off control circuit 17 closes the electromagnetic valve 7 by conducting the electric current to the electromagnetic valve breaker 18 for this time ΔT24. When the electromagnetic valve 7 is closed, the elastic blood supply tube 6 through which the blood flows is press-closed and blood flow is interrupted for time ΔT24. This time ΔT24 is the myocardial systole; and once this time is over, the electromagnetic valve 7 opens and blood is allowed to reflow through the elastic blood supply 6. The time for which this blood flow is supplied is the ventricular diastole. By repeating the above-described operation, a diastolic augmentation effect is realized. It should be understood that the symbol 24 in FIG. 5 for “ΔT (electromagnetic valve current conduction time interval; delay time setting)” and the symbol 25 for “time of current conduction to the electromagnetic valve (pulse signal)” are interchangeable.

The operation is summarized below.

Blood removed from the femoral vein of patient H is sent through the blood removal lines (1 and 2) to the centrifugal pump 3. Centrifugal force is applied by the centrifugal pump 3 to the blood when the turning body thereof that is in contact with the blood is turned, and the blood is sent to the artificial lung 5 (oxygenator) by this centrifugal force. The blood that has been oxygenated by the artificial lung 5 is returned, through the elastic blood supply tube 6 and through the port of electromagnetic valve 7, to femoral artery 9 of the patient H. Blood supply is stopped and started at this time by the opening and closing of the electromagnetic valve 7, in other words, by pressing to close and releasing to open the blood supply tube 8. As a result, pulsatile flow is obtained. The time relationship in which this pulsatile flow is generated is such that blood flows at times other than when blood flow is interrupted for time ΔT24 (25), from the rise in R wave 22 of the electrocardiogram waveform 20 as the starting point until ventricular systole is over. The pulsatile flow generation control means 11 that realizes this pulsatile flow generation comprises the amplification circuit 14 for electrocardiogram waveform 20, the waveform shaping circuit 15 including an R wave detection circuit and a circuit for generating a trigger (pulse) with the starting point being this R wave 22 (21), the delay pulse generation circuit 16 (delay circuit) for receiving trigger input and generating a pulse having ΔT time interval 24, and the electromagnetic valve breaker 18 for receiving the pulse from the delay circuit 16 and conducting or interrupting the electric current flowing to the electromagnetic valve breaker 18 (and electromagnetic valve 7). Actually, during the pulse generation period (25) of ΔT (24), the electric current to the electromagnetic valve 7 is conducted so as to close the electromagnetic valve 7; and as a result, the blood supply tube 8 is pressed to close and blood flow is interrupted. Blood flow is interrupted by ΔT24 of one heart beat period, and at all other times the blood flow is sent through femoral artery 9 of the patient. The time for which this blood flows is the ventricular diastole and a diastolic augmentation effect can be realized and participate in the recovery of the affected myocardium.

It should be noted that the pulsatile flow referred above is the one that generates blood that flows together with a pulsatile flow produced by the heart of patient H, and it is intended to raise blood pressure additionally when there is a reduction in blood pressure during diastole of the heart itself, to promote the blood flow to the coronary artery, and to realize a diastolic augmentation effect. When sufficient diastolic blood pressure is not obtainable because the affected heart has heart disease, this augmentation effect is realized and recovery of the heart is promoted.

Moreover, in the electromagnetic valve 7 described above, the valve closes only when electric current is conducted to the coil that actuates the electromagnetic valve 7; and when no electric current is conducted, pressure is not generated at the valve, and the valve is released to open under the restoring force of the elastic tube (elastic blood supply tube 6). This is the reason for using an elastic tube.

Amplification circuit 14 in FIG. 5 is an amplification circuit for amplifying the electrocardiogram waveform, and the amplification rate is variable (amplification adjustment operation means 111 in FIG. 2). This amplification circuit can be easily realized using an operational amplification circuit. The size of the electrocardiogram waveform voltage varies from individual to individual and can be easily processed after adjusting the amplification rate; therefore, the amplification rate is adjusted using amplification circuit 14 such that the input waveform R wave 22 (21) inputted to waveform shaping circuit 15 is sufficiently increased.

Waveform shaping circuit 15 is a circuit for detecting R wave 22 and generating the input pulse to the delay circuit 16 and can be realized using a comparator circuit (R wave detection circuit). Peak voltage varies with the patient H; therefore, threshold 21 of R wave 22 can be set as needed such that R wave 22 can be efficiently detected (R wave sensitivity adjustment operation means 112 in FIG. 2).

The input pulse to the delay circuit 16 that has been outputted from the waveform shaping circuit 15 is converted to delay pulse of time interval ΔT24 (25) using the delay circuit 16 (T wave delay adjustment operation means 113 in FIG. 2). This time interval ΔT24 (25) representing the cardiac systole is dependent on heart beat period T of the patient H, and because it varies from individual to individual, time 25 is artificially changed. A variety of methods are considered for realizing this delay circuit 16. For instance, it can be realized using timer 555, which is an IC element.

Voltage of, for instance, 15 W or higher is needed to operate the electromagnetic valve 7, and electric current of 0.6 A or greater must be fed from a 24V direct electric current power source to the electromagnetic valve 7. Therefore, a relay element (electromagnetic valve breaker 18) is used in order to control the conduction and interruption of the electric current. Relay (18) is a type of switch, and pulse (25) of ΔT (24) opens and closes the relay (18). During time interval (24) of ΔT, the relay (18) is closed. When the relay (18) is closed, electric current is fed to the electromagnetic valve 7; the tube 6, which feeds auxiliary blood flow, or “pulsed blood flow obtained using the present apparatus Y (X)”, is press-closed; and the auxiliary blood flow is interrupted.

When there is no input of pulse (25) of ΔT (24), the relay (18) is interrupted; the electric current to the electromagnetic valve 7 is stopped; the arterial blood supply tube 8 that was press-closed is opened; and auxiliary blood flows into aorta 9 of the patient H. This time is cardiac diastole, and a diastolic augmentation effect is realized.

The electrocardiogram waveform 20 amplification rate, R wave detection threshold 21, and cardiac systole ΔT24 (25) can be set by the pulsatile flow generation control means 11 (the above-described control box operating systems 111, 112, 113) as needed such that the pulsatile flow generation control means 11 will respond appropriately to changes in the heart beat period and cardiac systole as well as the electrocardiogram waveform 20 of the patient H.

As previously explained, the present invention provides physiological pulsatile flow to a body by mechanical means when auxiliary circulation is being conducted for cardiac therapy, and is of importance to the field of medicine in that improvement of therapeutic effects can be expected.

### Embodiment 2

Moreover, the clinical effect of the system of the present invention (including the pulsatile flow generation control device) was confirmed by laboratory animal experiment.

### Object of Experiment

Today, percutaneous pulmonary support is often used as the therapeutic method of first chose for acute cardiac dysfunction. Nevertheless, the conventional PCPS system uses a centrifugal pump for a variety of reasons. Furthermore, because such treatment is indicated for cases in which the heart itself is in a state of dysfunction, there are times when sufficient pulsatile flow is not obtained and auxiliary circulation is maintained in a state of steady flow. Therefore, by means of the structure of the present invention, the inventors developed an apparatus for generating a pulsatile flow capable of electrocardiogram synchronization using a simple electromagnetic valve (a pulsatile flow generation control device), and its functionality and safety were confirmed.

### Method of Experiment

The specimens were white rabbits (male: 2.5 to 3.0 kg, n (number of specimens)=3).

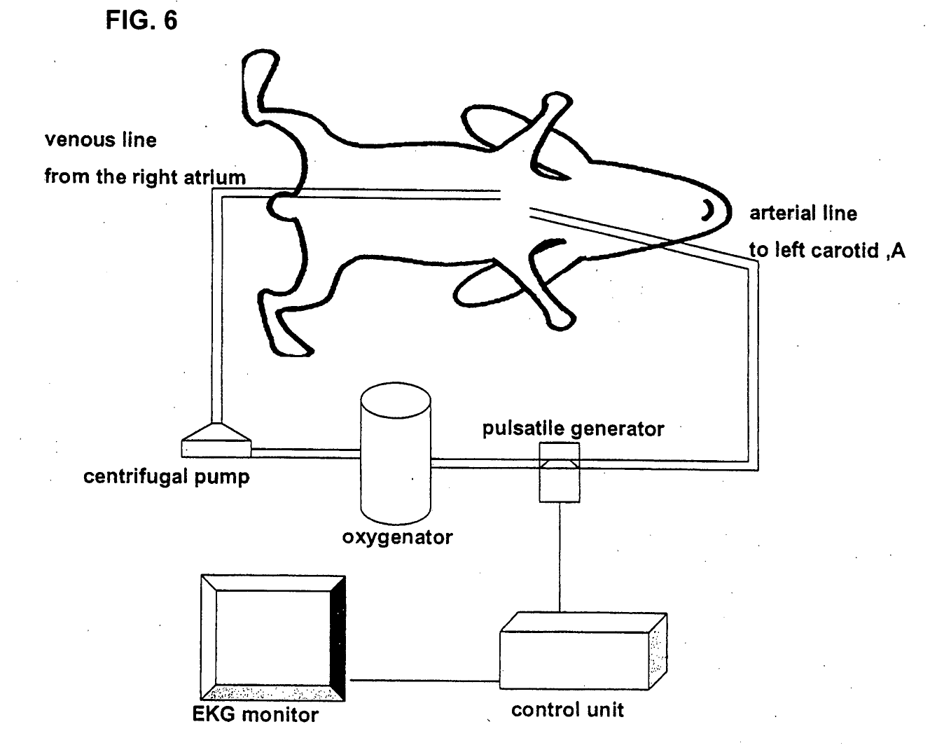

FIG. 6 shows a circuit diagram of the system of the present invention (including pulsatile flow generation control device) used in the present experiment. The system includes the following elements:
Venous line from the right atrium: Blood removal (venous) line from the right atrium
Arterial line to left carotid A: Blood supply (arterial) line to the left carotid artery
Centrifugal pump
Oxygenator (Artificial lung)
Pulsatile generator: Pulsatile flow generator
Control unit
EKG monitor

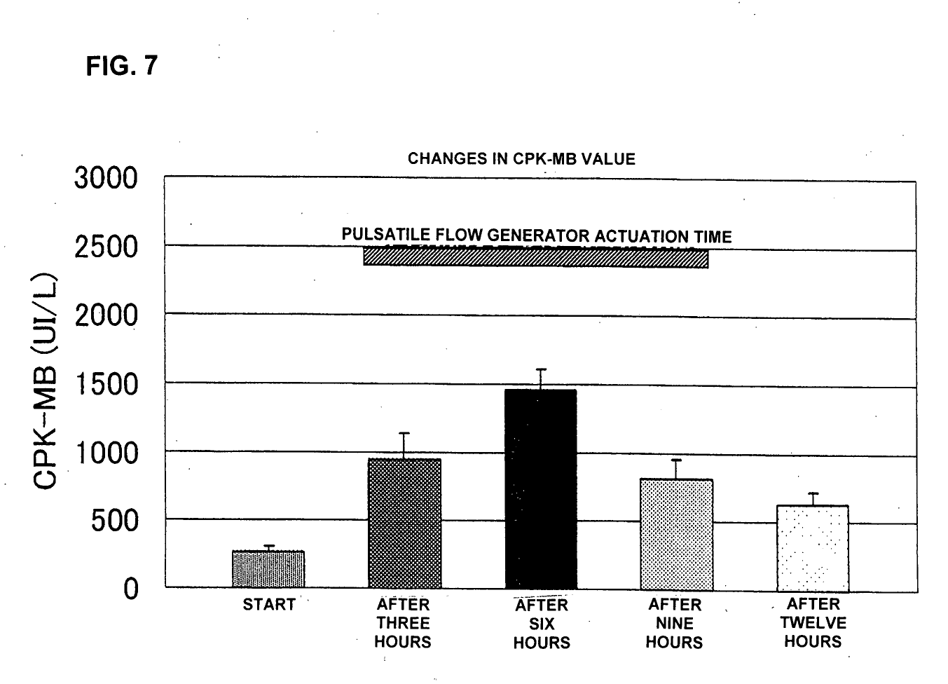

Here, auxiliary circulation was performed for 12 hours in each example (specimen) and the quantity of flow was controlled in synchronization with the electrocardiogram diastole. During the experiment, the CPK-MB fraction (creatinine phosphokinase MB fraction) was periodically measured and cardiac function was evaluated. FIG. 7 is a graph showing changes in the CPK-MB value.

### Results and Discussion of Experiment

Once the experiment was over, electron microphotographs (not appended) of the site press-closed by the electromagnetic valve of the pulsatile flow generator were taken where the tube had been subjected to three hours or six hours of continuous valve actuation. When damages to the inside surface of the circuit (tube) at these places of continuous actuation were evaluated, no damage was found, confirming that there are no problems with safety.

With respect to the experimental results, electrocardiogram synchronization was possible in all cases, and a rise in the systolic pressure could be confirmed. This indicates that the present invention is also useful in improving cardiac function.

### Embodiment 3

Furthermore, the system of the present invention (including the pulsatile flow generation control device) is not limited to the applications in Embodiments 1 and 2 and is thus a general-purpose system. For instance, a pulsatile flow state, which is a physiological fluid property, can be easily obtained while supplying myocardial protective liquid for myocardial protective infusion during routine heart surgery.

### Object of Experiment

Therefore, a myocardial protection experimental circuit that incorporated the pulsatile flow generation control device was formed and the fluid properties of pulsatile flow myocardial protection were confirmed.

### Method of Experiment

A pillow having a capacity of 2.0 mL was attached to a commercial myocardial protection circuit, the pulsatile flow generator of the present invention was actuated, and the pre-pillow (A) and post-pillow (B) maximum pressure/minimum pressure were measured. Commercial myocardial protective fluid (Saint Thomas II fluid) was used as the fluid.

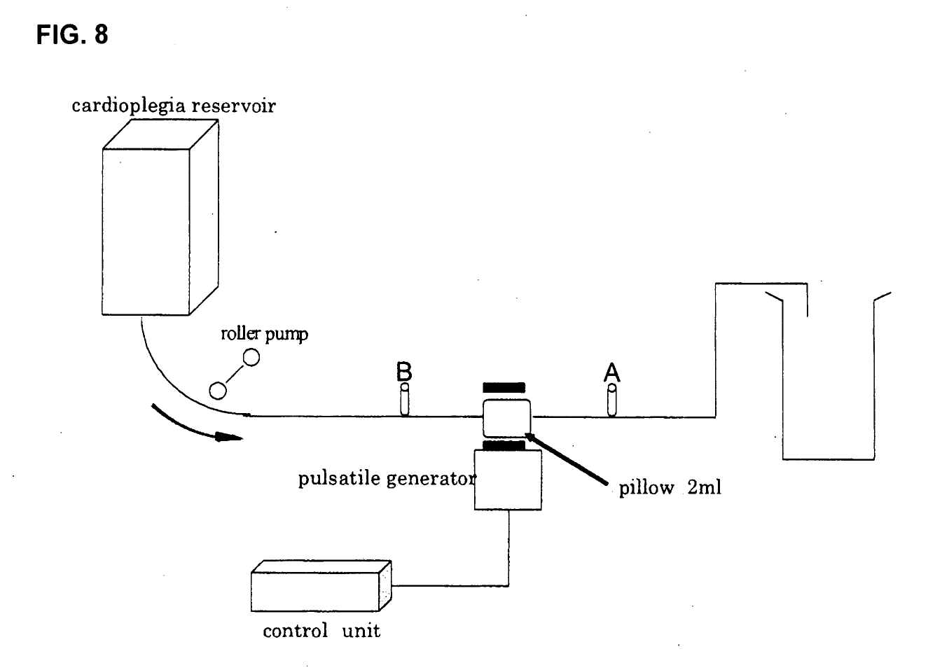

FIG. 8 shows an explanatory diagram of the pulsatile flow myocardial protection experimental circuit. The circuit comprises the following elements:
Cardioplegia reservoir: Reservoir
Roller pump
Pillow
Pulsatile generator: Pulsatile flow generator
Control unit

### Results and Discussion of Experiment

The measurement results in Table 1 are the average of measuring blood ten times, rounded off to a decimal point or less. It should be noted that (a) in Table 1 is the control data and shows the case of steady flow (pulsatile flow generator OFF).

TABLE 1
Steady flow (pulsatile flow generator OFF)

| | Maximum pressure/minimum pressure |
|---|---|
| | (pulse pressure) mmHg |
| A · B | 50/30 (20) |

Pulsatile flow (pulsatile flow generator ON)

Number of pulses (pulses/minute)

| | 60 | 45 | 30 | 15 |
|---|---|---|---|---|
| A | 90/40 (50) | 75/40 (35) | 90/40 (50) | 80/40 (40) |
| B | 100/70 (30) | 80/40 (40) | 80/40 (40) | 75/40 (35) |

(a) Myocardial protective fluid infusion by roller pump (liquid temperature of 5° C., flow speed of 250 mL/min)
(b) Measured pressure for each number of times pulse was actuated by pulsatile flow generator (liquid temperature of 5° C., 250 mL/min, tube press-closed for 500 ms)

As shown in Table 1, pulse pressure of 20 mmHg was able to be confirmed, even with a conventional myocardial protection system using a roller pump (a). On the other hand, it was possible to generate a maximum pulse of 50 mmHg by pulsatile myocardial protective infusion using the structure of the present invention (b). Moreover, generation of gas in the circuit was not seen during actuation; therefore, it was confirmed that the present invention can be used for myocardial protective infusion.

As is clear from these experimental findings, use of the means of the present invention to supply myocardial protective fluid is promising in terms of improvement in myocardial protective effects.

### INDUSTRIAL APPLICABILITY

The present invention is capable of supplying a physiological pulsatile flow which is synchronized with an electrocardiogram during auxiliary circulation therapy performed for cardiac dysfunction.

Moreover, throughout extracorporeal circulation during routine heart surgery, it is possible to supply a pulsatile flow, which was previously only possible in a state of cardiac arrest.

Furthermore, it is also possible to supply a physiological pulsatile flow for the myocardial protection that today is performed by steady flow and for selective cerebral perfusion performed for treatment of thoracic aorta aneurysm; therefore, the present invention is also useful in terms of myocardial protection and cerebral protection.

1. A pulsation-type auxiliary circulation system used in a blood circulation system comprising
a blood circulation path that is externally connected to a human body in cardiac dysfunction (a patient) and comprised of a blood removal line and a blood supply line; and
a centrifugal pump for using the blood circulation path as an auxiliary circulation path or for circulating blood between a human body and an artificial lung; wherein
said pulsation-type auxiliary circulation system conducts or promotes heart disease therapy by generating pulsatile flow in synchronization with the systole and diastole of the patient's own pulse based on measurement of the electrocardiogram waveform and/or pressure waveform, and comprises
an electromagnetic valve disposed in the blood supply line so as to be on a delivery side of the artificial lung,
a control unit for performing control-output relating to opening and closing operation of the electromagnetic valve, and
a biological signal monitoring device that is connected to the control unit and that is capable of measuring and displaying biological signals including at least the electrocardiogram waveform and pressure waveform; and
said control unit has a pulsatile flow generation control means for periodically interrupting a supply of blood by maintaining the electromagnetic valve closed for a predetermined time interval via at least a delay circuit.

2. The pulsation-type auxiliary circulation system according to claim 1, wherein
the predetermined time interval is a delay time setting of the delay circuit that performs detection and cancellation for blocking of the blood supply line by electromagnetic valve operation;
the delay time setting is time from when delay is detected with threshold electrocardiogram R waveform as a starting point until when delay is canceled with beginning of electrocardiogram T waveform generation as an ending point; and
the delay cancellation can be adjusted while monitoring dichrotic notch of the pressure waveform, which is a distinguishing waveform when aortic valve is obstructed.

3. A pulsatile flow generation control device used in a blood circulation system comprising
a biological signal monitoring device that is externally connected to a human body in cardiac dysfunction (a patient) and capable of measuring and video-display-outputting an electrocardiogram waveform and/or pressure waveform,
a blood circulation path comprised of a blood removal line and a blood supply line, and
a centrifugal pump for using the blood circulation path as an auxiliary circulation path or for circulating blood between a human body and an artificial lung; wherein
said pulsatile flow generation control device is a pulsatile flow generation control device wherein auxiliary circulation therapy is conducted or promoted by generating pulsatile flow in synchronization with systole and diastole of patient's own pulse based on measurement of electrocardiogram waveform and/or pressure waveform, and comprises
an electromagnetic valve disposed in the blood supply line so as to be on a delivery side of an artificial lung, and
a control means for performing control-output relating to opening and closing operation of the electromagnetic valve; and
the control means has at least a delay circuit and actuates the electromagnetic valve to close based on delay detection, maintains the electromagnetic valve closed for a predetermined time interval, and actuates the magnetic valve to open based on delay cancellation such that a supply of blood is periodically interrupted.

4. The pulsatile flow generation control device according to claim 3, wherein
said control means includes an electrocardiogram waveform amplification circuit, a waveform shaping circuit, a delay circuit, an electromagnetic valve current on/off control circuit, and a device for opening and closing the electromagnetic valve; and
said control means performs control-output for inputting electrocardiogram R waveform detected from amplified electrocardiogram waveform as a starting point and performing delay detection, actuating the electromagnetic valve to close based on delay detection, maintaining the electromagnetic valve closed for a predetermined time interval; and actuating the electromagnetic valve to open based on delay cancellation.

5. The pulsatile flow generation control device according to claim 3, wherein
the predetermined time interval is a delay time setting of the delay circuit that performs detection and cancellation for blocking of the blood supply line by electromagnetic valve operation;
the delay time setting is time from when delay is detected with threshold electrocardiogram R waveform as a starting point until when delay is canceled with beginning of electrocardiogram T waveform generation as an ending point; and
the delay cancellation can be adjusted while monitoring dichrotic notch of the pressure waveform, which is a distinguishing waveform when aortic valve is obstructed.

6. A pulsatile flow generation control method used in a blood circulation system comprising
a blood circulation path that is externally connected to a human body in cardiac dysfunction (a patient) and comprised of a blood removal line and a blood supply line; and
a centrifugal pump for using the blood circulation path as an auxiliary circulation path or for circulating blood between a human body and an artificial lung; wherein
said pulsatile flow generation control method
conducts or promotes heart disease therapy by generating pulsatile flow in synchronization with systole and diastole of patient's own pulse based on measurement of electrocardiogram waveform and/or pressure waveform, and
performs control-output in which the electrocardiogram waveform obtained from patient is amplified, electrocardiogram R waveform is detected as a trigger by waveform shaping therapy, a starting point is inputted to a delay circuit, an electromagnetic valve is actuated to close and is maintained closed for a predetermined time interval based on delay detection, and the electromagnetic valve is actuated to open based on delay cancellation.

7. The pulsatile flow generation control method according to claim 6, wherein
the predetermined time interval is a delay time setting of the delay circuit that performs detection and cancellation for blocking of the blood supply line by electromagnetic valve operation;
the delay time setting is time from when delay is detected with threshold electrocardiogram R waveform as a starting point until when delay is canceled with beginning of electrocardiogram T waveform generation as an ending point; and
the delay cancellation can be adjusted while monitoring dichrotic notch of the pressure waveform, which is a distinguishing waveform when aortic valve is obstructed.

8. The pulsatile flow generation control device according to claim 4, wherein
the predetermined time interval is a delay time setting of the delay circuit that performs detection and cancellation for blocking of the blood supply line by electromagnetic valve operation;
the delay time setting is time from when delay is detected with threshold electrocardiogram R waveform as a starting point until when delay is canceled with beginning of electrocardiogram T waveform generation as an ending point; and
the delay cancellation can be adjusted while monitoring dichrotic notch of the pressure waveform, which is a distinguishing waveform when aortic valve is obstructed.
